(19) United States
(12) Patent Application Publication
(10) Pub. No.: US 2011/0268711 A1
(43) Pub. Date: Nov. 3, 2011

(54) PANCREATIC STELLATE CELL SPECIFIC PROMOTER AND USES THEREOF

(75) Inventors: Lang Zhuo, Singapore (SG); Zhaobing Ding, Singpore (SG)

(73) Assignee: AGENCY FOR SCIENCE, TECHNOLOGY AND RESEARCH, Connexis (SG)

(21) Appl. No.: 12/674,414

(22) PCT Filed: Aug. 21, 2008
(86) PCT No.: PCT/SG2008/000305

§ 371 Date: Sep. 17, 2010

Related U.S. Application Data
(60) Provisional application No. 60/935,599, filed on Aug. 21, 2007.

(51) Int. Cl. A61K 48/00; A61P 1/18; C12N 5/10; C12N 15/85; C12Q 1/68

(52) U.S. Cl. 424/93.21; 435/455; 435/6.13; 435/6.1; 435/325; 435/375; 514/44 R; 514/44 A

(57) ABSTRACT

There is presently provided a method for effecting pancreatic stellate cell-specific gene expression comprising delivering a nucleic acid comprising a glial fibrillary acidic protein promoter operably linked to a coding sequence to a pancreatic stellate cell.

### CROSS REFERENCE TO RELATED APPLICATIONS

This application claims benefit of, and priority from, U.S. provisional patent application No. 60/935,599, filed on Aug. 21, 2007, the contents of which are fully incorporated herein by reference.

### FIELD OF THE INVENTION

This invention relates generally to pancreatic stellate cell specific promoters for gene expression and methods and uses thereof.

### BACKGROUND OF THE INVENTION

Pancreatic fibrosis is a pathological feature associated with chronic injuries, inflammation, and tumour of the pancreas. In contrast to liver fibrosis, the underlying etiology of fibrogenesis in the pancreas had been poorly understood until the identification of stellate cells in the pancreas (PSCs) as possible effector cells of excessive extra-cellular matrix (ECM) production (Bachem, M. G., et al., Gastroenterology, 1998, 115(2): 421-32; Apte, M. V., et al., Gut, 1998, 43(1): 128-33). It is increasingly being recognised that PSCs are a major cell type involved in mediating pancreatic fibrosis. Therefore, targeting PSCs may represent a superior therapeutic strategy for the treatment of pancreatic diseases, such as pancreatitis, pancreatic fibrosis, and pancreatic cancer.

PSCs are estimated to comprise about 4% of pancreatic cells in rat, and are located in the interacinar and interlobular regions of the pancreas. Isolated primary stellate cells from the pancreas rapidly trans-differentiate from a quiescent state to an activated myofibroblast morphology in culture, characterized by the loss of vitamin A fat droplets, the accumulation of alpha smooth muscle actin (α-SMA) and ECM proteins, such as collagen and fibronectin. This in vitro culture system is likely to, at least in part, mimic the PSC activation in vivo during pancreatic fibrosis (Omary, M. B., et al., J Clin Invest, 2007, 117(1): 50-9). Recent intensive characterizations of pancreatic stellate cells have provided evidence for the role of PSCs in pancreatic fibrosis (Omary et al., supra; Ellenrieder, V., et al., Rocz Akad Med Bialymst, 2004, 49: 40-6). Subsequent studies in animal model and human patients with chronic pancreatitis also indicated that α-SMA-positive PSCs are the main production source of collagen in fibrotic tissues (Luttenberger, T., et al., Lab Invest, 2000, 80(1): 47-55; Haber, P. S., et al., Am J Pathol, 1999, 155(4): 1087-95; Casini, A., et al., J Pathol, 2000, 192(1): 81-9; Shek, F. W., et al., Am J Pathol, 2002, 160(5): 1787-98; Neuschwander-Tetri, B. A., et al., Lab Invest, 2000, 80(2): 143-50). Moreover, studies on interactions between activated PSCs and cancer cells implicate PSCs in the progression of pancreatic cancer (Schneiderhan, W., et al., J Cell Sci, 2007, 120(Pt 3): p. 512-9).

At the molecular level, key cytokines (such as TGF-β, PDGF, TNF-α) and signaling pathways (such as MAPK family, PI3 kinase, TGF-β/SMAD pathway, RHO kinase, JAK/STAT, activator protein-1, NF-kappaB, PPARγ), have been implicated in PSC activation (McCarroll, J. A., et al., Biochem Pharmacol, 2004, 67(6): 1215-25; Masamune, A., et al., World J Gastroenterol, 2005, 11(22): 3385-91; McCarroll, J. A., et al., Gut, 2006, 55(1): 79-89; Mews, P., et al., Gut, 2002. 50(4): 53541; Jaster, R., et al., Gut, 2002. 51(4): 579-84; Jaster, R., Mol Cancer, 2004, 3: 26; Masamune, A., et al., Br J Pharmacol, 2003, 140(7): 1292-302; Masamune, A., et al., Tohoku J Exp Med, 2003, 199(2): 69-84; Fitzner, B., et al., Int J Colorectal Dis, 2004, 19(5): 414-20). Mechanisms that interrupt these signaling pathways offer potential antifibrogenic drugs, but may also result in side effects to numerous other cell types, since these signaling pathway do not specifically target PSCs (Pearson, G., et al., Endocr Rev, 2001, 22(2): 153-83; Derynck, R. and Y. E. Zhang, Nature, 2003, 425(6958): 577-84). Thus, methods that specifically target PSCs in the pancreas may provide a preferred approach to treating disorders and diseases related to pancreatic fibrosis.

### SUMMARY OF THE INVENTION

In one aspect, the present invention provides a method for effecting pancreatic stellate cell (PSC)-specific gene expression, the method comprising delivering a nucleic acid comprising a glial fibrillary acidic protein (GFAP) promoter operably linked to a coding sequence to a PSC.

In another aspect, the present invention provides a pancreatic stellate cell (PSC) comprising a nucleic acid molecule comprising a coding sequence operably linked to glial fibrillary acidic protein (GFAP) promoter, the coding sequence encoding a marker molecule or a therapeutic molecule.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate by way of example only, embodiments of the present invention:

FIG. 1: The 2.2-kb human glial fibrillary acidic protein (hGFAP) promoter directed expression of the DsRed reporter gene in pancreatic cell lines. Transient transfection was carried out in 6-well culture plates, 4 μg of GFAP-DsRed plasmids were transfected with LIPOFECTAMINE 2000™ according to the manufacturer's instructions and the cells were visualized by fluorescent microscopy after 24 hours. A: SAM-K cells, B: SIPS cells, C: LTC-7 cells, D: LTC-14 cells, E: ARIP ells, F: Rin-m cells. Scale bar=30 μm for all panels of FIG. 1;

FIG. 2: The 2.2-kb hGFAP promoter directed expression of the DsRed and lacZ reporter genes in primary mouse PSCs and pancreatic tissue. (A) Oil red O staining of primary PSCs 48 hours after primary PSCs isolation. Arrow indicates fat droplets. (B) Immuno-staining of α-SMA of primary PSCs after 8 days in culture. High amounts of stress-fiber was observed in the highly activated PSCs. Nuclei were counterstained with DAPI. (C) Transient transfection of primary PSCs with GFAP-DsRed construct. DsRed expression was visualized by fluorescent microscope. (D and E-F) X-gal staining of pancreatic tissue sections prepared from GFAP-LacZ transgenic mice. The arrow head indicates an islet, whereas the arrows indicate acinar cells. The only positively stained cells are PSCs (blue) (β-Gal was targeted to the nucleus in the GFAP-LacZ transgenic mice). (F) A representative pancreatic tissue section subject to the same X-gal staining displayed no blue signal. Scale bar for (A) to (E)=30 μm and for (F)=300 μm;

FIG. 3: Lack of transgene response to exogenous cytokines. SAM-K cells stably expressing the GFAP-LacZ transgene were treated with TGF-β (1 ng/ml, 10 ng/ml), PDGF-BB (10 ng/mI) and TNF-α (10 ng/ml) separately, and the transgenic β-Gal activity was measured at different time points using an assay kit from Promega (WI, USA). No significant change in enzymatic activity was observed for any of the treatment, suggesting that the potential effect of the exogenous cytokines could be overwhelmed by the autocrine cytokines in the activated PSCs;

FIG. 4: The GFAP-lacZ transgene was regulated by inhibitors of MAPK and PI3 kinases. SAM-K cells stably expressing the GFAP-LacZ transgene were with 10 μM of each kinase inhibitors separately (LY 294002 as an PI3 kinase inhibitor, SB 203580 as an p38 kinase inhibitor, U0126 as an ERK inhibitor, SP 600125 as an JNK inhibitor). The transgenic β-galactosidase activity was measured at 24 hours and 48 hours. 0.2% DMSO was used as a vehicle control (*P<0.001); and

FIG. 5: The GFAP-lacZ transgene expression was inhibited by Vitamin A and its metabolites. SAM-K cells stably expressing the GFAP-LacZ transgene were treated with 2 μM of retinol or 5 μM of its metabolites. The transgenic β-galactosidase activity was measured at 24 hours and 48 hours after the treatment. 0.2% of DMSO was used as vehicle control.

### DETAILED DESCRIPTION

Cell-specific promoters are of great interest to for their potential to drive expression of a target gene in a specific subpopulation or subset of cells either in vitro or in vivo. Cell-specific promoters are one of the primary means through which specialized cellular functions are limited to a particular differentiated cell type. The ability of these promoters to direct transcription of associated genes is regulated by the intracellular concentrations and activities of transcription factors in a specific type of cells.

Cell-type specific gene expression using a cell-specific cellular promoter to restrict gene expression in targeted tissues is a useful technique in biological studies to investigate cellular functions of a gene, as well as in medical applications to eliminate side effects caused by off-target expression of a therapeutic gene.

Currently, tissue- or cell-type specific promoters are employed to control specificity of expression of a transgene. This strategy is especially appealing to expression of a suicide gene in cancer treatment, which employs either toxic genes or genes encoding enzymes that turn prodrugs into toxic compounds.

Specific targeting of pancreatic stellate cells offers a potential treatment for pancreatic fibrosis and disorders that involve or arise from pancreatic fibrosis, including pancreatitis and pancreatic cancer.

To date, there has been no appropriate promoter that can be used for specific targeting of PSCs in the pancreas. Here, it is disclosed that the glial fibrillary acidic protein (GFAP) promoter can target PSCs in vitro and in vivo for specific gene expression, and that the promoter responds to multiple signaling stimuli in the activated PSCs.

The inventors have found that the GFAP promoter, including a 2.2-kb region of the human GFAP promoter (hGFAP), directs specific expression in PSCs in vitro and in vivo, including in immortalized and primary PSCs as well as in PSCs in the pancreas of transgenic GFAP lacZ mice. By using different kinase inhibitors, vitamin A and its metabolites, the inventors also found that the GFAP promoter in PSCs is regulated by multiple intracellular signaling pathways implicated in the activation of PSCs. Thus, the GFAP promoter may be used to specifically target PSCs for anti-pancreatic fibrosis research and therapy.

GFAP is an intermediate-filament protein, expressed exclusively in cells of glial origin (Eng, L. F., J Neuroimmunol 1985, 8: 203-14). In astrocytes, a fragment of the human GFAP (hGFAP) promoter has been shown to drive expression of operatively coupled transgenes in vitro and in vivo. The activity of this promoter fragment in non-astrocytic cells has been shown to be less predictable. The promoter fragment unreliably expressed lacZ in Müller cells in transgenic mice lines, leading to the suggestion that Müller cells may require regulatory elements beyond those contained in the promoter fragment (Brenner (1994), J Neurosci. 14: 1030). In Schwann cells, the transcription initiation site of the endogenous GFAP promoter is 169 nucleotides upstream from the transcription initiator site in astrocytes (Feinsten et al. (1992) J. Neurosci Res. 32(1):1). Further, while Schwann cells are known to express endogenous GFAP, these cells are also unreliably labeled in hGFAP-LacZ transgenic mice (Zhuo (1997), Developmental Biology 187:36).

Thus, there is presently provided a method for effecting PSC-specific gene expression, the method comprising delivering into a PSC a nucleic acid molecule comprising a GFAP promoter operably linked to a coding sequence.

The term “cell” (including in the context of “PSC”) as used herein refers to and includes a single cell, a plurality of cells or a population of cells where context permits, unless otherwise specified. Similarly, reference to “cells” also includes reference to a single cell where context permits, unless otherwise specified.

The PSC may be any pancreatic stellate cell, including an in vitro PSC, including primary or immortalised, and an ex vivo PSC explanted from a subject and may be a quiescent or activated PSC. The PSC may be a transgenic PSC as described herein in an in vivo context, for example an animal model comprising a transgenic PSC, or an endogenous PSC in an in vivo context, including in a human subject.

The PSC may be, in particular embodiments, a SAM-K, a SIPS, an LTC-7 or an LTC-14 cell, or may be a primary PSC obtained from an FVB/N mouse.

The GFAP promoter may be any promoter that directs expression of glial fibrillary acid protein, including a mammalian GFAP promoter. In one embodiment, the GFAP promoter comprises the 2.2 kb 5′ region flanking the human GFAP gene, as described in Zhuo et al., Developmental Biology, 1997, 187: 36-42 and in Brenner et al., J Neurosci, 1994, 14(3 Pt 1): 1030-1037, more specifically, the 2.2 kb fragment corresponding to nucleotides −2163 to +47 (SEQ ID NO: 1) of the hGFAP gene (Accession Number M67446).

In particular embodiments, the glial-specific promoter region comprises, consists essentially of or consists of the sequence of the human GFAP promoter or the rat GFAP promoter, as shown below:

Human GFAP Promoter [SEQ ID NO: 1]:

GTCTGCAAGCAGACCTGGCAGCATTGGGCTGGCCGCCCCCCAG
GGCCTCCTCTTCATGCCCAGTGAATGACTCACCTTGGCACAGACACAATG
TTCGGGGTGGGCACAGTGCCTGCTTCCCGCCGCACCCCAGCCCCCCTCAA
ATGCCTTCCGAGAAGCCCATTGAGTAGGGGGCTTGCATTGCACCCCAGCC
TGACAGCCTGGCATCTTGGGATAAAAGCAGCACAGCCCCCTAGGGGCTG
CCCTTGCTGTGTGGCGCCACCGGCGGTGGAGAACAAGGCTCTATTCAGCC
TGTGCCCAGGAAAGGGGATCAGGGGATGCCCAGGCATGGACAGTGGGTG
GCAGGGGGGGAGAGGAGGGCTGTCTGCTTCCCAGAAGTCCAAGGACACA
AATGGGTGAGGGGACTGGGCAGGGTTCTGACCCTGTGGGACCAGAGTGG
AGGGCGTAGATGGACCTGAAGTCTCCAGGGACAACAGGGCCCAGGTCTC
AGGCTCCTAGTTGGGCCCAGTGGCTCCAGCGTTTCCAAACCCATCCATCC
CCAGAGGTTCTTCCCATCTCTCCAGGCTGATGTGTGGGAACTCGAGGAAA
TAAATCTCCAGTGGGAGACGGAGGGGTGGCCAGGGAAACGGGGCGCTGC
AGGAATAAAGACGAGCCAGCACAGCCAGCTCATGCGTAACGGCTTTGTG
GAGCTGTCAAGGCCTGGTCTCTGGGAGAGAGGCACAGGGAGGCCAGACA
AGGAAGGGGTGACCTGGAGGGACAGATCCAGGGGCTAAAGTCCTGATAA
GGCAAGAGAGTGCCGGCCCCCTCTTGCCCTATCAGGACCTCCACTGCCAC
ATAGAGGCCATGATTGACCCTTAGACAAAGGGCTGGTGTCCAATCCCAG
CCCCCAGCCCCAGAACTCCAGGGAATGAATGGGCAGAGAGCAGGAATGT
GGGACATCTGTGTTCAAGGGAAGGACTCCAGGAGTCTGCTGGGAATGAG
GCCTAGTAGGAAATGAGGTGGCCCTTGAGGGTACAGAACAGGTTCATTC
TTCGCCAAATTCCCAGCACCTTGCAGGCACTTACAGCTGAGTGAGATAAT
GCCTGGGTTATGAAATCAAAAAGTTGGAAAGCAGGTCAGAGGTCATCTG
GTACAGCCCTTCCTTCCCTTTTTTTTTTTTTTTTTTTTTTGTGAGA
CAAGGTCTCTCTCTGTTGCCCAGGCTGGAGTGGCGCAAACACAGCT
CACTGCAGCCTCAACCTACTGGGCTCAAGCAATCCTCCAGCCTCAGCCT
CCCAAAGTGCTGGGATTACAAGCATGAGCCACCCCACTCAGCCCTTT
CCTTCCTTTTTAATTGATGCATAATAATTGTAAGTATTCATCATGGT
CCAACCAACCCTTTCTTGACCCACCTTCCTAGAGAGAGGGTCCTCTT
GATTCAGCGGTCAGGGCCCCAGACCCATGGTCTGGCTCCAGGTACCAC
CTGCCTCATGCAGGAGTTGGCGTGCCCAGGAAGCTCTGCCTCTGG
GCACAGTGACCTCAGTGGGGTGAGGGGAGCTCTCCCCATAGCTGGG
CTGCGGCCCAACCCCACCCCCTCAGGCTATGCCAGGGGGTGTTGCCA
GGGGCACCCGGGCATCGCCAGTCTAGCCCACTCCTTCATAAAGCCCT
CGCATCCCAGGAGCGAGCAGAGCCAGAGCAT

Rat GFAP Promoter [SEQ ID NO: 2]:

CCTGCAGGGCCCACTAGTCTGTAAGCTGGAAGTCTGGCAGTGC
TGAGCTGGCCAACCCCCTCAGGACCTCCTCCTTGTGCCCACTGAATGACT
CACCTTGGCATAGACATAATGGTCAGGGGCGGGCACACAGCCTGATTCC
CGCTGCACTCCAGGCCCCCTTCAATGCTTTCCGAGAAGTCCATTGAGCTG
GGAGCTTGTACTGCACCAAGGGCTGACATCCTGGCAGCCAGGGATGAAA
GCAGCCCATGGGGCTACCCTTGCCGTATGCCTCACTGGCGGCAGAGAAC
AAGGCTCTATTCAGCAAATGCCCTGGAGTAGACACCAGAAGTCCAAGCA
TGGGCAGAGGAAGGCAGGCGTTGGGGGCTGGAGGGGAGCAGAGCTGTCT
GTTTTCCAGAAGCCCAAGGGTACAGATGGCGCCTGGGGGGGAACTGAGT
GGAGGGGATAGATGGGCCTGAGATCTCAAACATCAACAGCCTCCTCCCC
ACCAACGATGAAGGTGGAGGTTGGTTTCCCAGACCTACATATCCCCCAGA
GACCTGGTGTATGAAAATTCAAAGGAGGTAAGTCTCCTGAGAGAACGGG
GGGCTCACAAATGAAGCCAGCTGTCTTACCCTATCAGGACCTACGTGCAT
TCCTTCTGTCCTGCCCCCTAAACACACAGCCAGAGGCTCAAATTGATTCT
GGAGTCACAAAGGGGGCTTGAAACCCCAGCCCCCCACTCCTGAACTCCA
GGAATGAGAAGATAGTATTGGAGGGGTTCAGAGGAGAGGGCTCTGCACA
TCTGTTGAGAATGGGGGTCCCAGGAGAGTGTAATTTAGGCTGATCCCGGA
GGAAGGGAATAGGCTCTTCAAGATCCTAGCATCTCACAGGCCCACAGAG
AAGTTCAGAGTTGGGGCAGCCCTGGCTTACAGGCTCTAAGAACTGGAGG
CAGTTTACCCAACCCAGCTGTGTGCATGCTGTCCCTCTCTCTGTCTCTGTC
TGTCTCTCTCTGTCTCTGTCTCTCTGTGTGTGTGTGTGTGTGCTCACACAC
GTGTGTGTTTATCACACAAATGTTCATGTGTGTGTACATACATGTGTTGA
GGCCAGAGGTCAACCTCAGACACTGTTGACTTGGTTGTATGAGATAACAT
TTCCCCCTGGGACCTGGGATTTGCCAATTAGTGTGACCCAGGAAGCCTAC
TTATTTTCATTCCTCAGCACTGCAGTTACAAGTATGCACTGTCAAACCAG
GCCTTTTTTTTTTTTTTTTTCCAAACCAGGCCTTTTGTATTCGCTCTG
TGGCTAGAACTTGGGTCTCCATGCTTGACAGGCAAGCGATTTATGGA
CTAAGCTGTTTCCTCGGCCCTCTCTTGACCCATTTACCAGAAATGGG
GTTTCCTTGATCAATGGTTAAGCCAGGCTGGTGTTCCCAGGAAACCCT
TGACTCTGGGTACAGTGACCTTGGTGGGGTGAGAAGAGTTCTCTCCA
TAGCTGGGCTGGGGCCCAGCTCCACCCCCTCAGGCTATTCAATGGGGT
GCTGCCAGGAAGTCAGGGGCAGATCCAGTCCAGCCCGTCCCTCAATAAA
GGCCCTGACATCCCAGGAGCCAGCAGAAGCAGGGCAT

As used herein, “consists essentially of” or “consisting essentially of” means that the nucleic acid sequence includes one or more nucleotide bases, including within the sequence or at one or both ends of the sequence, but that the additional nucleotide bases do not materially affect the function of the nucleic acid sequence, for example to function as a promoter to drive expression of an operably linked coding sequence in a PSC.

In other embodiments, the GFAP promoter is a promoter having at least 80%, at least 85%, at least 90%, at least 95%, or at least 99%, sequence identity to SEQ ID NO: 1 or SEQ ID NO: 2, while still retaining the ability to direct PSC-specific gene expression.

In other embodiments, the GFAP promoter is any naturally occurring or engineered sequence that is an allelic variant or derivative of the 2.2 kb hGFAP sequence. As used herein, allelic variants or derivatives contemplate sequences that contain one or more nucleotide additions, substitutions and deletions while retaining the ability to direct selective transgene expression in PSC cells. For example, in different embodiments, the GFAP promoter may correspond to smaller fragments of the 2.2 kb hGFAP promoter that retain the ability to direct PSC-specific expression. Allelic variants of the hGFAP promoter also include naturally occurring homologous sequences from other organisms or allelic variants or derivatives thereof that retain the ability to direct PSC-selective transgene expression.

Methods for identifying allelic variants or derivatives directing PSC-specific expression would be known to a person skilled in the art, for example, by deletion mapping.

In still other embodiments, the GFAP promoter is a recombinant hybrid promoter comprising one or more heterologous enhancer elements operably coupled to all or a portion of the 2.2 kb hGFAP promoter sequence, or an allelic variant or derivative thereof, provided the hybrid promoter is capable of selectively directing expression in a PSC. In this context, selective expression refers to a promoter that can direct the expression of an operably linked coding sequence in a PSC but not in non-PSC pancreatic cells such as, for example, acinar cells or islet cells.

The enhancer elements in the hybrid promoter may be selected from known elements, such as, for example, enhancer elements from the human cytomegalovirus, or may be novel elements identified thorough known methods, such as, for example, enhancer trap assays.

Hybrid promoters may be synthesized using standard molecular biology and molecular cloning techniques known in the art, for example, as described in Sambrook et al. (2001) Molecular Cloning: a Laboratory Manual, 3^{rd} ed., Cold Spring Harbor Laboratory Press). As will be understood, the term “recombinant” when referring to a nucleic acid molecule or construct means that heterologous nucleic acid sequences have been recombined, such that reference to a recombinant nucleic acid molecule refers to a molecule that is comprised of nucleic acid sequences that are joined together or produced by means of molecular biological techniques.

In various embodiments, GFAP promoter variants and derivatives may be substantially homologous in that they hybridize to all or part of the hGFAP promoter under moderate or stringent conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH. Stringent hybridization may, for example, be conducted in 5×SSC and 50% formamide at 42° C. and washed in a wash buffer consisting of 0.1×SSC at 65° C. Washes for stringent hybridization may, for example, be of at least 15 minutes, 30 minutes, 45 minutes, 60 minutes, 75 minutes, 90 minutes, 105 minutes or 120 minutes or longer.

The degree of homology between sequences may also be expressed as a percentage of identity when the sequences are optimally aligned, meaning the occurrence of exact matches between the sequences. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence alignment may also be carried out using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). In various embodiments, the variants and derivatives may be at least 50%, at least 80%, at least 90% or at least 95%, or at least 99% identical to SEQ ID NO: 1 or SEQ ID NO: 2 as determined using such algorithms, while still retaining the ability to direct PSC-specific gene expression.

The GFAP promoter is operably linked to a coding sequence that encodes a product to be expressed within the PSC.

A first nucleic acid sequence is operably linked with a second nucleic acid sequence when the sequences are placed in a functional relationship. For example, a coding sequence is operably linked to a promoter if the promoter activates transcription of the coding sequence. Similarly, a promoter and an enhancer are operably linked when the enhancer increases the transcription of operably linked sequences. Enhancers may function when separated from promoters and as such, an enhancer may be operably linked to a promoter even though it is not contiguous to the promoter. Generally, however, operably linked sequences are contiguous.

The coding sequence is any nucleic acid sequence that encodes a product that can be expressed under control of an operably linked promoter. For example, the coding sequence may encode a protein or may encode an RNA molecule such as siRNA.

In various embodiments, the coding sequence encodes a marker molecule that may be useful for the identification of a PSC in vitro and/or in vivo. A marker molecule is any molecule whose expression may be detected and used to determine that PSC specific expression has been effected under control of the GFAP promoter.

The marker molecule may be an RNA molecule, whose expression may be determined by known method based on nucleic acid hybridization or amplification, such as for example, RT-PCR.

Alternatively, the marker molecule is a marker protein. As used herein, “marker protein” refers to a protein whose expression and/or subcellular localization may be readily detected, such as, for example, a fluorescent protein, including green fluorescent protein (GFP). DNA vectors encoding fluorescent proteins, for example, blue, cyan, green, and yellow-green and red are commercially available (Clontech). Other marker proteins would be known to a person skilled in the art. In different embodiments, the marker protein may be an enzyme whose expression may be readily determined by providing a specific substrate and detecting the products of enzymatic turnover. Examples of enzymatic marker proteins include, for example, β-galactosidase and luciferase. Other enzymatic marker proteins would be known to a person skilled in the art. In other embodiments, the marker protein may be any protein whose expression may be detected immunologically, for example, by providing a labeled antibody that specifically recognizes and binds the marker protein, or a fragment thereof. The antibody may be a polyclonal antibody or a monoclonal antibody and may be directly or indirectly labeled according to methods known in the art, such as, for example, labeling with a fluorescent dye and detecting expression of the marker protein by fluorescence microscopy. Other immunological-based detection methods, including, for example, immunogold staining, radiolabelling, and colourometric enzymatic precipitation would be known to a person skilled in the art. In specific embodiments, the marker protein is β-galactosidase.

In other embodiments, the transgene operably linked to the GFAP promoter encodes a therapeutic product. As used herein, “therapeutic product” includes any product that, once expressed, has a therapeutic effect, including an RNA or protein that is involved in disease prevention, treatment, or a RNA or protein that has a cell regulatory effect that is involved in disease prevention or treatment.

The therapeutic product may be any therapeutic product that may be involved in the treatment of a pancreatic fibrosis related disease or disorder. A pancreatic fibrosis related disease or disorder refers to any disease, disorder or condition which may cause, result in, or is associated with pancreatic fibrosis, including a primary retinopathy or a secondary retinopathy including, for example, pancreatitis, pancreatic cancer.

As used herein, “treat” or “treatment” in respect of a pancreatic fibrosis related disorder refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). “Treating” can also mean prolonging survival of a subject beyond that expected in the absence of treatment. “Treating” can also mean inhibiting the progression of disease, slowing the progression of disease temporarily, although more preferably, it involves halting the progression of the disease permanently.

The therapeutic product may be a polypeptide, including a protein or a peptide, a ribozyme, an siRNA, an antisense RNA or a microRNA.

In various embodiments, the therapeutic product is an anti-fibrotic polypeptide. Various anti-fibrotic polypeptides would be known to a person skilled in the art. Without being limited to any particular theory, the anti-fibrotic peptide may reduce or inhibit fibrosis by: (a) reducing inflammation to avoid stimulating PSC activation, such as, for example TNF-β, PDGF-BB or TNF-α antagonists and interleukin-10 (IL-10); (b) directly down-regulating PSC activation, such as for example, γ-interferon and hepatocyte growth factor (HGF); (c) neutralize proliferative, fibrogenic, contractile or pro-inflammatory responses of stellate cells, such as, for example, antagonists to PDGF, FGF or TGFα, including soluble cognate receptor fragments; (d) induce PSC apoptosis such as, for example, Bcl-xL or Fas; or (e) induce ECM degradation, such as, for example, matrix metalloprotease-8 (MMP-8).

In a specific embodiment, the therapeutic product is IL-10. Other therapeutic products would be known to a person skilled in the art and include, for example, the products of the Smad 7 gene, and the product of dominant negative alleles of Smad 3, Smad 4 and TGFR. Dominant negative forms of the Smad proteins are known and may be created by serine to alanine substitutions in the phosphorylation site, for example, by replacing SSXS sites to AAXA, where X represents any amino acid. Dominant negative forms of TGFR would also be known to a person skilled in the art. Other therapeutic products include, for example, dominant negative alleles of the platelet-derived growth factor receptor (PDGFR) and Diptheria toxin.

As would be understood by a person skilled in the art, a “dominant negative allele” is an allele whose expression inhibits or reduces the biological effect of the expression product of a wild-type allele.

In other embodiments, the therapeutic product is a siRNA. siRNAs are generally double stranded 19 to 22 nucleotide sequences that can effect post-transcriptional silencing of cognate mRNAs, allowing for selective suppression of gene expression. Generally, and without being limited to any specific theory, the sequence of one strand of the siRNA therapeutic product will be complementary to a portion of the mRNA of the gene sought to be silenced. For example, the siRNA may be designed to hybridize with mRNA encoding TGF-β1. PSC cells are the most important source of TGF-β1 in pancreatic fibrosis and inhibiting TGF-β may inhibit matrix production and accelerate its degradation (Freidman (2003), J Pancreatol. 38 Suppl 1:S38). In other embodiments, the siRNA may be designed to hybridize against α1(I) collagen mRNA. Increased α1(I) collagen expression in PSCs has been shown to be mediated primarily through a post-transcriptional mechanism, with the half life of α1(I) collagen mRNA increasing from 1.5 hours in quiescent cells to greater than 24 hours in activated PSCs. In yet other embodiments, the siRNA may be designed to hybridize against nucleic acids encoding platelet-derived growth factor (PDGF) or extracellular matrix molecules, such as, for example, fibronectin, laminin and integrin.

Guidelines for designing siRNAs would be known to the person skilled in the art, or siRNA designed to hybridize to a specific target may be obtained commercially (Ambion, Qiagen). For example, siRNAs with a 3′ UU dinucleotide overhang are often more effective in inducing RNA interference (RNAi). Other considerations in designing siRNAs would be known to a person skilled in the art.

The operably linked GFAP promoter and coding sequence may be contained within the context of a larger nucleic acid molecule. The larger nucleic acid molecule may be, for example, a vector, including an expression vector for delivery to and stability in an expression system for use in an appropriate expression system that can support transcription from a GFAP promoter. For example, the operably linked GFAP promoter and coding sequence may be included in a plasmid, chromosome, mitochondrial DNA, plastid DNA, virus, or nucleic acid fragment.

The vector may contain additional elements that allow for the integration, selection, replication or manipulation of the vector. For example, the vector may contain a selection marker, such as, for example, the neo and Zeo^{R} genes, which may confer cellular resistance to G-418 and Zeocin™, respectively. Other selection markers would be known to a person skilled in art. Other additional elements would also be known to a person skilled in the art and include, for example, a replication sequence, a multiple cloning site (MCS), and a transcription termination sequence, such as, for example, bovine growth hormone poly-adenylation signal sequence (BGH pA).

The vector may be linear or circular, and, if circular, may be supercoiled. As would be known to a person skilled in the art, the efficiency of chromosomal integration may be enhanced by providing a linear vector, whereas episomal transfection may be more efficient with supercoiled DNA. Linear vectors may be prepared from circular vectors by known methods, for example, by cutting with an appropriate restriction endonuclease.

Vectors containing the operably linked GFAP promoter and coding sequence may be synthesised using known vectors and using standard molecular biology and molecular cloning techniques to generate recombinant nucleic acid molecules, as described above.

The operably linked GFAP promoter and coding sequence, including in a vector, is delivered into a PSC.

As used herein, “delivery” or “delivering” into a PSC refers to any process wherein the exogenous nucleic acid molecule is introduced into a PSC, and includes viral and non-viral methods. Examples of viral methods would be known to a person skilled in the art and include, for example, the administration of lentiviral vectors, adenoviral vectors, adeno-associated viral vectors and baculoviral vectors. Non-viral transfection methods would also be known to the skilled person and include, for example, naked plasmids, DEAE-dextran, calcium phosphate co-precipitation, microinjection, electroporation, nucleofection (Amaxa), liposome-mediated transfection, non-liposomal lipid preparations, cationic lipids, and polycationic polymers. Many of the non-viral transfection reagents and protocols are commercially available and would be known to a person skilled in the art. In specific embodiments, delivery may be effected by LIPOFECTAMINE™ 2000 (Invitrogen) according to the directions provided by the manufacturer. Delivery may also be effected by FUGENE™ 6 (Roche) according to the directions provided by the manufacturer.

A person skilled in the art would know how to confirm that the operably linked GFAP promoter and coding sequence has successfully been delivered into a PSC. For example, where the operably linked GFAP promoter and coding sequence is included in a nucleic acid molecule encodes a selectable marker conferring cellular resistance against a drug, cells containing the nucleic acid molecule may be identified by exposing cells to that drug. Stable transgenic PSC lines may be selected by increasing the concentration of the drug and maintaining the transfected cells at the higher drug concentrations for an appropriate period of time, which will depend, among other things, on the proliferative rate of the cells. In some instances, the stable transfected cell lines may be obtained after at least 6 weeks of drug selection.

Alternatively, where the nucleic acid molecule encodes a fluorescent marker protein, for example, GFP or any of its fluorogenic derivatives, cells containing the nucleic acid molecule may be identified and selected by optical methods, such as, for example, fluorescent activated cell sorting (FACS) (Yata et al. (2003), Pancreatology. 37:267). Other methods of identifying PSCs containing the nucleic acid molecule include methods based on nucleic acid hybridization, such as, for example, southern analysis and/or PCR amplification, would be known to a person skilled in the art.

Once delivered into the cell, the expression of the coding sequence is “under control” of the GFAP promoter in the PSC, meaning that the GFAP promoter is operably linked to the coding sequence and is the promoter that directs transcription of the coding sequence. Thus, factors that activate or enhance transcription from the GFAP promoter may result in increased expression of the coding sequence, and factors that inhibit or block transcription from the GFAP promoter may result in decreased expression of the coding sequence, relative to expression in the absence of any such factors.

As used herein, “expression” or “expressing” refers to production of any detectable level of a product encoded by the coding sequence.

As will be understood by a person skilled in the art, transcription levels may be determined by methods that measure the amount of RNA expressed, for example, Northern blot, quantitative RT-PCR, RNA run-on assays, gene chip analysis. Alternatively, transcription levels may be measured by measuring the optical, colorimetric, fluorogenic, immunogenic or enzymatic properties of a protein product encoded by the coding sequence. For example, the activity of a reporter gene, such as β-galactosidase, may be determined by known assays using the chromogenic substrate 5-Bromo-4-Chloro-3-Indolyl-BD-Galactopyranoside (X-gal). Alternatively, transgene expression may be determined by known immunological methods, for example, Western analysis.

The above method for effecting PSC-specific gene expression may be used to identify fibrogenesis modulating agents involved in pancreatic fibrosis. Fibrogenesis modulating agents include agents that increase, upregulate or activate fibrogenesis, as well as agents that decrease, downregulate or inhibit fibrogenesis. Thus, fibrogenesis modulating agents include pro-fibrotic agents as well as anti-fibrotic agents.

In one embodiment the method allows for identification of anti-fibrotic agents. Thus, the method additionally comprises detecting the expression levels of a gene that is operably linked to a GFAP promoter in a PSC, in the presence and the absence of a putative anti-fibrotic agent.

The two expression levels are then compared and anti-fibrotic agents are identified where the expression of the gene is reduced or abolished in the presence of the agent. Expression levels may be detected by known methods, such as, for example, by the methods described herein for detecting expression of a coding sequence.

In the present method, the coding sequence operably linked to the GFAP promoter may be an endogenous gene encoding the natural GFAP protein. For example, the expression may be performed using a vector containing a region that includes the GFAP promoter and coding region. The expression may also be performed in a PSC using the endogenous GFAP promoter and coding region. The levels of expression of the endogenous GFAP gene may be detected using methods known in the art, including by detecting levels of mRNA, for example by Northern blot, quantitative RT-PCR, RNA run-on assays, gene chip analysis or by any other methods known in the art for detecting mRNA levels in a cell or in a cell-free expression system.

Alternatively, the coding sequence operably linked to the GFAP promoter may encode a marker molecule as described herein. For example, the expression may be performed using a vector containing the GFAP promoter operably linked to a reporter gene that encodes a detectable product, including a detectable mRNA or a detectable protein. Such an expression vector may be used in a cell free system or in a transgenic PSC that has been transfected with the expression vector. mRNA levels may be detected as described herein, or a protein product may be detected as described herein, including by immunoassay techniques such as immunoblots or ELISAs, by radioassays, by chemiluminescence assays, by optical methods, by colorimetric methods, by fluorogenic methods, or by enzymatic methods, or by other methods known in the art for detecting levels of transgenic products.

In a particular embodiment, the GFAP promoter is operably linked to the endogenous GFAP coding region, resulting in production of the GFAP mRNA transcript and ultimately the GFAP protein. In another particular embodiment, the GFAP promoter is operably linked to the lacZ reporter transgene, resulting in production of the β-galactosidase mRNA transcript and ultimately in the β-galactosidase protein. Detection of β-galactosidase is well known in the art, and kits for such detection are commercially available, including chemiluminescent assay kits (e.g. available from Roche, Mannheim, Germany).

The expression may be conducted in an in vitro cell-free expression system. Cell-free expression systems are known in the art and are commercially available, and typically include all of the required enzymes and substrates to produce a transcript and/or a protein, including an RNA polymerase that is capable of transcribing the GFAP promoter, ribonucleotides, ribozomes, amino acids, tRNAs. Conducting the present assay in a cell free system would allow for screening and identification of anti-fibrotic agents that directly interact with the GFAP promoter.

The compounds that are to be screened as putative anti-fibrotic agents may be added directly to the in vitro system, and the expression levels of the gene operably linked to the GFAP promoter are measured in the presence and absence of the putative anti-fibrotic agent. As stated above, an anti-fibrotic agent will result in measurable decreased expression from the GFAP promoter as compared to expression in the absence of the anti-fibrotic agent.

However, it may be desirable to identify anti-fibrotic agents that act indirectly on the GFAP expression levels, for example by acting further upstream in a pathway that activates GFAP expression. Thus, the expression may be performed in a cultured PSC.

In the method performed in a cell, the putative anti-fibrotic agent may be contacted with the cell, and expression levels of the gene operably linked to the GFAP promoter are then detected. As with the cell-free method, expression levels in the presence and in the absence of the putative anti-fibrotic agent are compared, with an anti-fibrotic agent providing measurably less expression than in the absence of the anti-fibrotic agent.

In certain embodiments, the PSC used is a SAM-K, a SIPS, an LTC-7 or an LTC-14 cell. The GFAP promoter may be the endogenous promoter operably linked to the endogenous GFAP coding sequence or the PSC may be transfected with a GFAP-lacZ expression construct.

The method may be performed using known pro-fibrotic agents to elevate the levels of expression from the GFAP promoter. The known pro-fibrotic agent may be, for example, TGF-β, PDGF-BB, TNF-α or lipopolysaccharide. The known pro-fibrotic agent may be administered to a cell in which the expression is to be performed, either prior to treatment with the putative anti-fibrotic agent, or concurrently with the putative pro-fibrotic agent.

For example, the PSC may be pre-treated with a pro-fibrotic agent for a given period sufficient to result in elevated expression from the GFAP promoter, for example from about 1 to about 3 days. The culture medium may then be replaced with fresh medium not containing any pro-fibrotic agent prior to treatment with the putative anti-fibrotic agent. Alternatively, the pro-fibrotic agent and the putative anti-fibrotic agent may be administered concurrently, for example by addition to the culture medium for a period from about 1 to about 3 days.

The putative anti-fibrotic agent may be administered to the PSC for example by addition to the culture medium, for a given time period, for example from about 2 hours, to about 3 days.

The present method may also be performed in a cell in an in vivo context, for example in an animal model including a transgenic animal model, by administering the putative anti-fibrotic agent to the animal and comparing expression levels from the GFAP promoter in PSCs in the animal given the putative anti-fibrotic agent with expression levels from the GFAP promoter in animals not given the putative anti-fibrotic agent.

Administration of or exposure to the putative anti-fibrotic agent may be done in comparison with administration of a known anti-fibrotic agent. The known anti-fibrotic agent may be, for example, epigallocatechin gallate (EGCG), genistein or N-acetylcysteine.

The known anti-fibrotic agent may be used to develop a standard curve for measuring expression levels or reduction of expression levels in response to a given concentration of a known anti-fibrotic agent, as will be appreciated by a skilled person. For example, the known anti-fibrotic agent may be added to the culture medium at a final concentration of about 0.1 μM or greater, of about 100 μM or less, or from about 0.1 μM to about 100 μM. For example, a standard curve may include various concentrations of known anti-fibrotic agent such as 0.1 μM, 0.2 μM, 0.5 μM, 1 μM, 5 μM, 10 μM, 25 μM, 50 μM and 100 μM, or such as 0.013 μM, 12.5 μM, 5 μM, 10 μM, 20 μM and 40 μM.

The concentration of putative anti-fibrotic agent to use can then be determined in relation to such a standard curve, adjusting the concentration or amount used so as to fall within the limits of the standard curve. A person skilled in the art could routinely determine the concentration of the putative anti-fibrotic agent to be used in the screen according to the in vitro or in vivo screening method. The appropriate amount of the putative anti-fibrotic agent employed in the screen will depend, among other things, on the nature of the anti-fibrotic agent. A person skilled in the art would know to determine the appropriate concentration range, for example, by screening over two or more orders of magnitude and would appreciate that more potent anti-fibrotic agents would decrease expression levels of the transgene at a lower concentration compared to a less potent anti-fibrotic agent.

The present method for identifying putative anti-fibrotic agents may be used to determine the effect of concurrent treatment with two or more known or putative anti-fibrotic agents. The method may be performed as generally described herein, with the two or more agents being administered to the expression system either sequentially or concurrently to determine whether treatment with two or more anti-fibrotic agents has a synergistic effect that is greater than the additive effects of the two or more agents when administered independently to the expression system.

The above-described method may be adapted for high-throughput screening of a large number of putative anti-fibrotic agents. For example, cells may be cultured in large numbers of individual batches, for example in 96-well culture plates. In this way, the method may be adapted for multiple simultaneous screening methods to be performed, using known automated techniques to conduct assays in parallel.

Any anti-fibrotic agents identified in the above-described methods may be used in the treatment of a disease or disorder in which pancreatic fibrosis occurs, including pancreatitis or pancreatic cancer.

In another embodiment the method allows for identification of pro-fibrotic agents. Thus, the method comprises detecting the expression levels of a coding sequence that is operably linked to a GFAP promoter in a PSC, in the presence and the absence of a putative pro-fibrotic agent. The two expression levels are then compared and pro-fibrotic agents are identified where the expression of the coding sequence is increased in the presence of the agent. Expression levels may be detected as described herein.

As above, identification of pro-fibrotic agents may be performed in a cell-free system or in a cell, including in an isolated cell in culture or in a cell in an in vivo animal model.

Generally, identification of pro-fibrotic agents may be performed as described herein for screening for anti-fibrotic agents. A skilled person will understand that when comparing results with those obtained for a known agent, the known agent will be a known pro-fibrotic agent, for example transforming growth factor-β (TGF-β), platelet derived growth factor-BB (PDGF-BB), tumour necrosis factor-α (TNF-α) or lipopolysaccharide (LPS).

The above method for effecting PSC-specific gene expression may be used to diagnose pancreatic fibrosis in a subject or determining the prognosis of a subject having or being likely to develop pancreatic fibrosis.

The subject may be any subject who currently has a pancreatic fibrosis related disease or disorder, who is likely to develop such a disease or disorder or in whom such a disease or disorder is desired to be prevented, including a human subject.

Explanted PSCs obtained from the subject are cultured. Levels of expression of the endogenous hGFAP mRNA or endogenous hGFAP protein are then detected as is known in the art and as described herein, including using immunoassay methods to detect the GFAP protein, including in situ immuno-staining methods.

Alternatively, the PSCs from the subject and PSCs not associated with pancreatic fibrosis may be transfected with a vector comprising the GFAP promoter operably linked to a coding sequence, as described herein.

The levels of expression from the GFAP promoter may be compared to levels of expression from the GFAP promoter in a cell that is not associated with pancreatic fibrosis. Such a cell that is not associated with pancreatic fibrosis may be from a healthy individual or from a cell line that has GFAP expression levels that are not increased due to activation of the PSC. The levels of expression from the GFAP promoter may be higher in cells from a subject that has pancreatic fibrosis than in cells not associated with pancreatic fibrosis, and may be higher be in cells from a subject prone to or predisposed to developing pancreatic fibrosis.

The GFAP expression levels may be compared in the presence and absence of a known fibrogenesis modulating agent. The fibrogenesis modulating agent may be an anti-fibrotic agent or a pro-fibrotic agent as described herein. As will be understood, in cells from a subject who has, is predisposed to, or is likely to develop a disease or disorder caused by or characterized by the presence of pancreatic fibrosis, contacting the cell with a pro-fibrotic agent tends to result in increased expression from the GFAP promoter. Similarly, contacting the cells with an anti-fibrotic agent tends to result in decreased expression from the GFAP promoter.

The above method for effecting PSC-specific gene expression may be used in vivo to treat a pancreatic fibrosis related disorder. Thus, the method includes administering to a subject a nucleic acid comprising a coding sequence encoding a therapeutic product operably linked to a PSC-specific hGFAP promoter, according to various aspects of the invention. In specific embodiments, the hGFAP promoter is the 2.2 kb promoter fragment of SEQ ID NO:1. In certain embodiments, the therapeutic product is an anti-fibrotic molecule, and in specific embodiments is an anti-fibrotic polypeptide.

The subject is any subject in need of such treatment, including a mammal, and particularly a human subject.

To deliver the nucleic acid molecule specifically to PSCs, the nucleic acid may be delivered by methods known in the art. Methods for introducing the nucleic acid molecule into mammalian cells in vivo are known, and may be used to administer the nucleic acid vector of the invention to a subject. A nucleic acid may be delivered into a PSC by direct injection of DNA, receptor mediated DNA uptake, viral-mediated transfection or non-viral lipid based transfection. The nucleic acid vector may be administered by microparticle bombardments, for example, using a commercially available “gene gun” (BioRad).

The nucleic acid molecule is administered in such amounts to achieve the desired results, for example, the expression of the therapeutic product in PSCs. For example, the nucleic acid may be delivered in such amounts to express sufficient amounts of the therapeutic product which functions to alleviate, mitigate, ameliorate, inhibit, stabilize, improve, prevent, including slow the progression of the disorder, the frequency of treatment and the type of concurrent treatment, if any.

The nucleic acid molecule may be administered in the context of a PSC that comprises the nucleic acid molecule.

An effective amount of the nucleic acid molecule or PSCs may be administered to subject, using methods known in the art, including by surgical implantation or by injection in or near the subject's pancreas. The term “effective amount” as used herein means an amount effective, at dosages and for periods of time necessary to achieve the desired result, for example, to treat the specific disorder. The amount of nucleic acid molecule or the number of total PSCs to be administered will vary, depending on, among other things, the disorder or disease to be treated, the mode of administration, the age and health of the patient and the expression levels of the therapeutic product.

To aid in administration, the nucleic acid molecule or the PSC may be formulated as an ingredient in a pharmaceutical composition. The compositions may routinely contain pharmaceutically acceptable concentrations of salt, buffering agents, preservatives and various compatible carriers or diluents. For all forms of delivery, the nucleic acid molecule may be formulated in a physiological salt solution.

The proportion and identity of the pharmaceutically acceptable diluent is determined by chosen route of administration, compatibility with a nucleic acid molecule including a live virus when appropriate, or compatibility with a live cell, and standard pharmaceutical practice. Generally, the pharmaceutical composition will be formulated with components that will not significantly impair the biological properties of the nucleic acid or the live PSC. Suitable vehicles and diluents are described, for example, in Remington's Pharmaceutical Sciences (Remington's Pharmaceutical Sciences, Mack Publishing Company, Easton, Pa., USA 1985).

Solutions of the nucleic acid molecule or PSC may be prepared in a physiologically suitable buffer. Under ordinary conditions of storage and use, these preparations contain a preservative to prevent the growth of microorganisms, but that will not inactivate or degrade the nucleic acid molecule. A person skilled in the art would know how to prepare suitable formulations. Conventional procedures and ingredients for the selection and preparation of suitable formulations are described, for example, in Remington's Pharmaceutical Sciences and in The United States Pharmacopoeia: The National Formulary (USP 24 NF19) published in 1999.

The forms of the pharmaceutical composition suitable for injectable use include sterile aqueous solutions or dispersion and sterile powders for the extemporaneous preparation of sterile injectable solutions or dispersions, wherein the term sterile does not extend to any live virus or cell that may comprise the nucleic acid molecule that is to be administered. In all cases the form must be sterile and must be fluid to the extent that easy syringability exists.

In another embodiment, PSCs comprising an exogenous nucleic acid molecule comprising a coding sequence operably linked to GFAP promoter as described herein are also contemplated. The PSCs may be isolated, in vitro cells, or may be in vivo cells.

As used herein, “isolated” refers to transgenic PSCs in in vitro culture, in the presence or absence of other cell types.

The PSC according to different embodiments may be used to study fibrogenesis, pancreatic fibrosis or for developing anti-fibrosis gene therapies. In some embodiments, the PSC cell is a transgenic rat SAM-K, SIPS, LTC-7 or LTC-14 cell.

Kits and commercial packages containing the various nucleic acid molecule constructs described herein, including an expression vector containing a coding sequence encoding a therapeutic product operably linked to a promoter comprising a PSC-specific GFAP promoter or a PSC comprising such a nucleic acid molecule, or kits and commercial packages containing a pharmaceutical composition as described herein, are contemplated. Such a kit or commercial package will also contain instructions regarding use of the included nucleic acid molecule, PSC or pharmaceutical composition, for example, use to diagnose or treat a pancreatic-fibrosis related disorder, for example or for expressing an expression product in a PSC, including in accordance with the methods described herein.

The invention is further exemplified by the following non-limiting example.

### EXAMPLE 1

Materials and Methods

Cell culture and reagents: Four rat PSC cell lines, SAM-K and SIPS (generously provided by Dr. A. Masamune (Masamune, A., et al., World J Gastroenterol, 2003, 9(12): 2751-8; Satoh, M., et al., Tohoku J Exp Med, 2002, 198(1): 55-69), LTC-7 and LTC-14 (generously provided by Dr. G. Sparmann (Sparmann, G., et al., Am J Physiol Gastrointest Liver Physiol, 2004, 287(1): G211-9), were maintained in F-12 medium containing 10% FBS and antibiotics (penicillin 100 units/ml, streptomycin 100 μg/ml). ARIP and Rin-m were from American Type Cell Collection (ATCC, VA, USA). Cell cultures were maintained at 37° C. in a humidified CO₂ (5%) incubator. Cell cultural reagents were from Invitrogen (CA, USA); collagenase P, pronase, DNase were from Roche Applied Science (Mannheim, Germany); retinol, 9-cis retinoic acid (9-RA), all-trans retinoic acid (ATRA), and HISTODENZ™ were from Sigma (Missouri, USA); kinase inhibitors LY 294002 (PI3 kinase inhibitor), U0126 (ERK inhibitor), SP 600125 (JNK inhibitor), SB 203580 (p38 kinase inhibitor) were from Merck Bioscience (Darmstadt, Germany), recombinant TGF-β1, TNF-α, and PDGF-BB were from R&D (MN, USA).

Construction of the 2.2-kb hGFAP-DsRed transgene: The plasmid vector pcDNA 4/TO/LacZ (Invitrogen, CA, USA) was used as cloning backbone. DsRed fragment of pDsRed-Express-N1 vector (Clontech, CA, USA) was excised by BamHI and Hind III, and cloned into the same restriction sites in the pcDNA 4/TO/LacZ. The DsRed fragment replaced the LacZ coding sequences and the plasmid was designated as pcDNA-CMV-DsRed. Then the 2.2-kb hGFAP promoter from GFAP-GFPS65T (Zhuo, L., et al., Dev Biol, 1997, 187(1): 36-42) was excised by Bgl II and Hind III, and replaced the CMV TetO2 promoter in the pcDNA-CMV-DsRed plasmid with same restriction sites. This plasmid was named as GFAP-DsRed. The cloning junction sequences were confirmed by sequencing.

Transfection and isolation of stable cell lines: The 2.2-kb hGFAP-LacZ (Maubach, G., et al., World J Gastroenterol, 2006, 12(5): 723-30) or the 2.2-kb hGFAP-DsRed plasmid was used to transfect PSC lines, using LIPOFECTAMINE™ 2000 (Invitrogen, CA, USA) according to the manufacturer's instructions. DsRed expression was identified under fluorescence microscope (Leica, Wetzlar, Germany). To isolate the stable clones expressing GFAPLacZ in SAM-K cell line, 24 hours after transfection, the cell culture medium was changed into medium containing Zeocin (500 μg/ml, Invitrogen, CA, USA). The single clones were isolated by dilution in the 96 well plates, the positive β-galactosidase (β-Gal) clones were identified by β-Gal staining kit from Invitrogen (CA, USA). Stable clones were maintained in culture medium containing 250 μg/ml Zeocin.

Treatment of PSCs with cell signaling inhibitors: Before treatment with different factors, SAM-K cells stably expressing the GFAP-LacZ at 70-80% confluence were starved in 0.5% FBS overnight, TGF-β (1 ng/ml, 10 ng/ml), PDBF-BB (10 ng/ml), and TNF-α (10 ng/ml) treatments were carried out at different time points, PBS buffer (0.2%) treatment served as control. Kinase inhibitors (10 μM), including LY 294002, U0126, SP600125, SB 203580, retinol and its metabolites, including retinol, 9-RA, ATRA, were applied to the cell culture medium at both 2 μM and 5 μM concentrations. Treatment in 0.2% DMSO served as control. The transgenic β-Gal activity was quantified at various times following treatment.

Primary stellate cells isolation and culture: The procedures for animal studies were approved by the Institutional Animal Care and Use Committee (IACUC) and the Biomedical Research Council (BMRC) of Singapore. Pancreatic tissues isolated from FVB/N mice were minced with sharp scissors, then incubated at 37° C. for about 20 minutes in Gey's balanced salt solution (GBSS) containing 0.05% collagenase P, 0.02% pronase, 0.1% DNase. The digested solution was passed through nylon mesh (150 μm, BD Biosciences, CA, USA), the filtered solution was washed once and mixed with HISTODENZ™ (Sigma, Missouri, USA) to form a 13.2% HISTODENZ gradient, centrifugation step was done at 1400 g for 20 minutes. The fuzzy band just above the interface of HISTODENZ was collected and re-suspended in F-12 medium containing 10% FBS and antibiotics (penicillin 100 units/ml, streptomycin 100 pg/ml). The purity of primary cells was analyzed by vitamin A auto-fluorescence, immuno-staining with α-SMA (positive), RecA-I (negative) antibodies.

Oil red O staining and Immunostaining of α-SMA in primary PSCs: The PSCs cultured for one day after isolation were fixed in 4% paraformaldehyde (PFA) for 5 minutes, and washed twice with propylene glycol, stained in 0.7% oil red O (Sigma, Missouri, USA) solution in Propylene glycol for 5 minutes, then washed three times with 85% propylene glycol, finally counter-stained with Hematoxylin. Immunostaining of αSMA was carried out on PSCs 8 days after isolation as previously described (Maubach, G., et al., Biochim Biophys Acta, 2007, 1773(2): 219-31).

β-Gal histochemistry and quantitative β-Gal activity in cell extracts: The GFAP-LacZ transgenic mice, originally generated by Brenner et al. (Brenner, M., et al., J Neurosci, 1994, 14(3 Pt 1): 1030-7), were obtained from the Jackson Laboratory. The mice were genotyped with PCR using the following primers:

(forward)
[SEQ ID NO: 3]
5′-ACTCCTTCATAAAGCCCTCG-3′;
(reverse)
[SEQ ID NO: 4]
5′-AACTCGCCGCACATCTGAACTTCAGC-3′.

To obtain cryostat sections, the pancreas was removed from both GFAP-LacZ transgenic mouse and wild type mouse of the same FVB/N genetic background, and were fixed in 4% PFA fixative for 30 minutes. The fixed tissues were infiltrated overnight with 30% sucrose. Small pieces of tissue were mounted in OCT medium, and 14-μm sections were collected onto POLYSINE™ slides (Menzel GmbH & Co KG, Braunschweig, Germany). The expression of lacZ gene was detected using a staining kit from Invitrogen. The sections were stained with X-gal substrate overnight at room temperature, and subsequently counter-stained with eosin, prior to being mounted on slides with HYDROMOUNT™ (National diagnostics, Georgia, USA). To quantify the GFAP-LacZ expression, the β-Gal activity in the total cell extracts was measured with an enzymatic assay kit (Promega, WI, USA).

Statistical analysis: All quantitative results were presented as mean±SE; data were analyzed with Student t-test.

Results

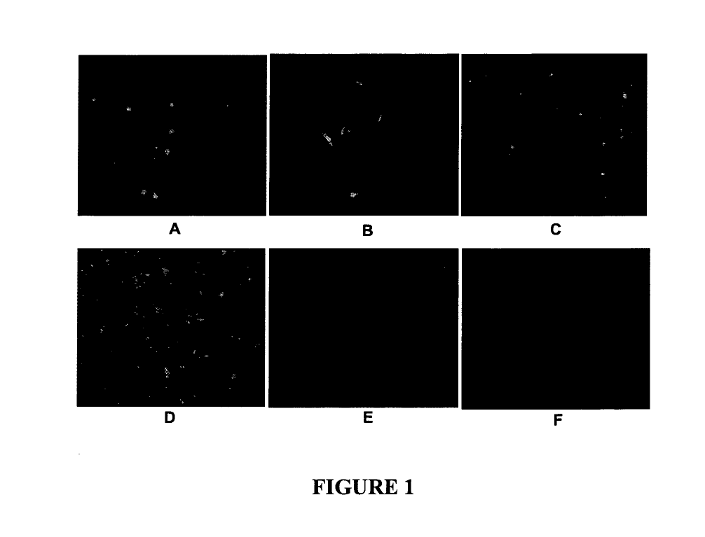

The hGFAP promoter directed reporter gene expressions specifically in PSCs in vivo and in vitro: Four different PSC lines from rat (SAM-K, SIPS, LTC-7, LTC-14) were used to test the GFAP transgene expression. Among them, SAM-K, LTC-7 and LTC-14 were immortalized by SV40 large T antigen, whereas the SIPS was derived via spontaneous immortalization. Early reports showed that SAM-K and LTC-7 were less activated than the SIPS and LTC-14 (Masamune, A., et al., supra; Satoh, M., et al., supra; Sparmann, G., et al., supra). In the present study, these four cell lines were all transiently transfected with the GFAP-DsRed construct. Fluorescent microscopy showed that all four PSC lines expressed the DsRed reporter (FIG. 1, A-D). In contrast, the same transgene failed to express in two non-PSC cell lines, i.e., an acinar cell line (ARIP from rat) and an insulin-producing β cell line (Rin-m from rat) (FIG. 1, E, F).

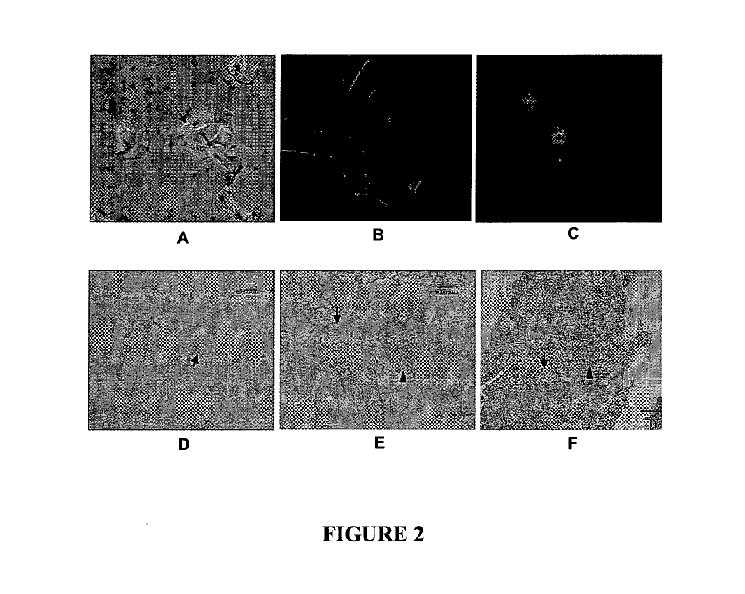

Since the transcriptional profiles of cell lines may change after immortalization, primary PSCs were isolated from mouse pancreas to test the transgene expression. Primary mouse PSCs with more than 95% purity, as confirmed by oil-red and α-SMA staining (FIG. 2A, B), were transiently transfected with the GFAP-DsRed plasmid. Fluorescent microscopy similarly confirmed the transgene expression in the primary PSCs (FIG. 2, C). However, the number of positive red cells was rather low, due to the low transfection efficiency of the plasmid-based vector in the primary PSCs.

To investigate the transgene expression in vivo, cryostat sections prepared from the pancreas of the GFAP-LacZ transgenic mice were stained with X-gal substrate. The staining results revealed many β-Gal positive (blue), stellate-like cells located in the inter-acinar space (FIG. 2, D, E), these cells are certainly not in the acinar (arrow) and islet (arrow head) region. None of β-Gal-positive cells was found in control wild type pancreatic tissue sections (FIG. 2, F). This indicated that the β-Gal staining is specific to PSCs in the transgenic mice.

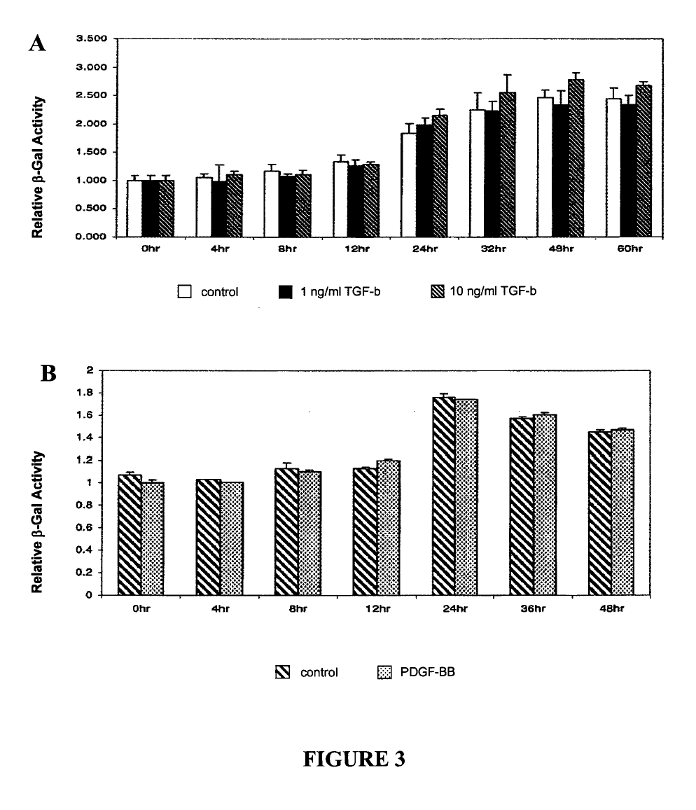

Minimal effect by pro-fibrogenic cytokines on GFAP-lacZ expression in a PSC line: Several key cytokines (TGF-β, PDGF-BB, and TNF-α) are known to play important roles in the activation of PSCs (Omary, M. B., et al., supra; Jaster, R., supra). To investigate whether these key pro-fibrogenic cytokines and signaling pathways could influence and cross-talk with GFAP transgene expression, SAM-K stably expressing GFAP-lacZ were treated with TGF-β (FIG. 3A), PDGF-BB (FIG. 3B) and TNF-α (FIG. 3C). None of the treatments specifically induced transgene expression (FIG. 3) at all time points (24, 32, 48 and 60 hours after treatments). For TGF-β, the absolute level of transgene expression in both the treated and the non-treated cells was elevated after 24 hrs, which may be due to the autocrine action of the pro-fibrogenic cytokines in a more confluent cultures.

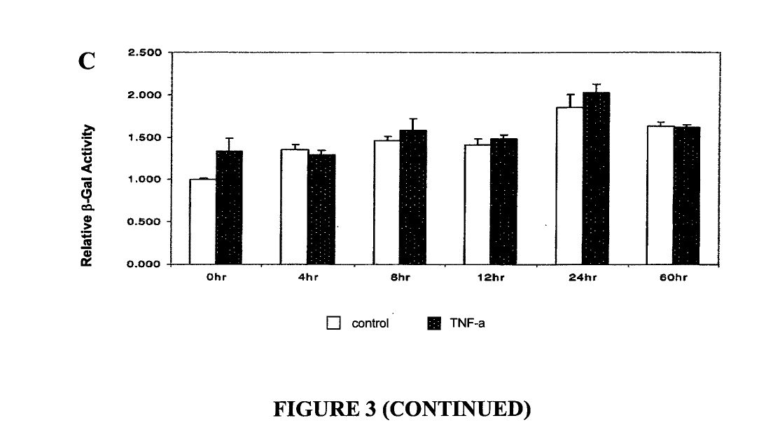

GFAP-lacZ transgene expression is down-regulated by MAP% and P13% inhibitors: Some signaling transduction pathways (MAPK and PI3 kinase) also play important roles in the activation of PSCs (Omary, M. B., et al., supra; Jaster, R., supra). Four kinase inhibitors were subsequently tested for their ability to influence the hGFAP transgene expression. After a preliminary trial, a concentration of 10 μM, at which no apparent cellular toxicity was observed, was selected for treatment with the four kinase inhibitors separately. Twenty-four hours after treatment, the ERK kinase inhibitor (U0 126) led to the biggest drop in 13-Gal activity, approximately 40% decrease in 13-Gal activity was achieved (P<0.001, FIG. 4). The INK inhibitor (SP600 125) and the P13-Kinase inhibitor (LY 294002) also had a significant effects on the 13-Gal activity, about 25%, 19% decreases in the 13-Gal activity, respectively (P<0.001, FIG. 4). However, the effect for p38 kinase inhibitor was not as significant (FIG. 4). Forty-eight hours after treatment, the ERKS inhibitor (U0126) continued to inhibit 13-Gal activity significantly (about 15% decrease in 13-Gal activity, P<0.01, FIG. 4), the same was also applied to INK inhibitor (SP 600125, about 10% decrease in 13-Gal activities, P<0.001, FIG. 4) and P13-Kinase inhibitor (LY 294002, about 15% decrease in 13-Gal activity, P<0.001, FIG. 4). The p38 kinase inhibitor (SB 203580) still had no significant effects on 13-Gal activity 48 hours after treatment (FIG. 4). The above results indicated the 2.2-kb hGFAP promoter is regulated by ERKs, INK, and P1-3 kinase pathways, not by p38 kinase pathway in pancreatic stellate cells.

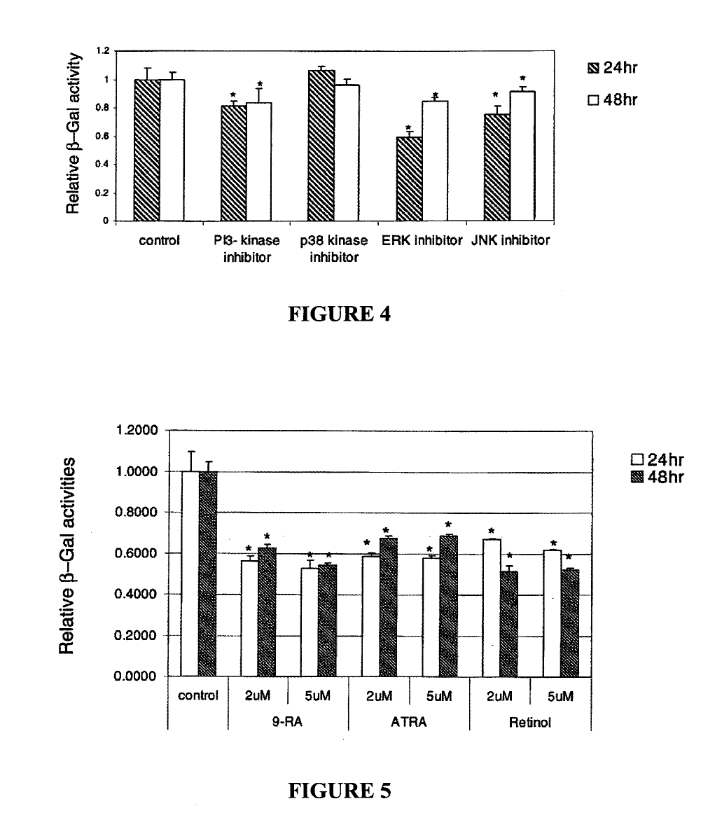

Inhibition of the hGFAP-lacZ transgene by retinol and its metabolites: To test the effect of Vitamin A on hGFAP promoter's activities, retinol and its metabolites ATRA and 9-RA were applied to the stable PSCs (SAM-K) expressing GFAP-LacZ. 24 hours after treatment, all treatments led to a significant decrease in transgene expression (FIG. 5): at 2 μM levels, the relative β-Gal activity for 9-RA, ATRA, and retinol treatments were decreased by about 44%, 42%, and 33%, respectively (P<0.001); at 5 μM levels, the relative β-Gal activities for 9-RA, ATKA, retinol treatments were decreased by about 48%, 42%, and 38%, respectively (P<0.001). 48 hours after treatment, all treatments χ◯ντινυεδ to have significant decreases of β-Gal activity (FIG. 5): at 2 μM levels, the relative β-Gal activities for 9-RA, ATRA, retinol treatments were decreased by 37%, 33% and 49%, respectively (P<0.001), at 5 μM levels, the relative β-Gal activities for 9-RA, ATRA, retinol treatment were decreased by about 46%, 31%, and 48%, respectively (P<0.001). Thus, regardless of concentration and length of treatment, all three treatments resulted in a significant drop of b-Gal activity. However, it should be noted that in previous studies higher concentrations (>10 μM) were toxic, leading to cell death (Pinzani, M., Gut, 2006, 55(1): 12-4).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for effecting pancreatic stellate cell (PSC)-specific gene expression, the method comprising delivering to a PSC a nucleic acid molecule comprising a glial fibrillary acidic protein (GFAP) promoter operably linked to a coding sequence, the GFAP promoter consisting essentially of the 2.2 kilobase region 5′ to a GFAP gene or a fragment thereof.

2. The method according to claim 1, wherein the GFAP promoter comprises the region from −2163 to +47 of a human GFAP promoter.

3. The method according to claim 1, wherein the GFAP promoter comprises the sequence set forth in SEQ ID NO: 1 or SEQ ID NO: 2, or an allelic variant of the sequence set forth in SEQ ID NO: 1 or SEQ ID NO: 2.

4. The method according to claim 1, wherein the GFAP promoter comprises a sequence having at least 80% sequence identity to SEQ ID NO: 1 or SEQ ID NO: 2, while still retaining the ability to direct PSC-specific gene expression.

5. The method according to claim 1 wherein the GFAP promoter sequence consists of the sequence set forth in SEQ ID NO: 1.

6. The method according to claim 1 wherein the coding sequence encodes a marker molecule or a therapeutic molecule.

7. (canceled)

8. The method according to claim 6 wherein the coding sequence encodes a polypeptide, a small interfering RNA or a microRNA.

9. The method according to claim 6 wherein the therapeutic molecule is an anti-fibrotic polypeptide.

10. The method according to claim 8 wherein the small interfering RNA is complementary to a portion of an mRNA encoding an extracellular matrix protein.

11. The method according to claim 6 that is a method of identifying a fibrogenesis modulating agent and wherein the coding sequence encodes a marker molecule, the method further comprising:
a) detecting a first expression level of the marker molecule in the PSC in the absence of a test compound;
b) detecting a second expression level of the marker molecule in the PSC in the presence of the test compound; and
c) comparing the first expression level and the second expression level,
whereby the first expression level greater than the second expression level indicates that the test compound is an anti-fibrotic agent and the first expression level less than the second expression level indicates that the test compound is a pro-fibrotic agent.

12. (canceled)

13. The method of claim 11 wherein the test compound is a putative anti-fibrotic agent and a pro-fibrotic agent is administered to the pancreatic stellate cell prior to said detecting a first expression level and said detecting a second expression level.

14. The method of claim 13 wherein the pro-fibrotic agent is TGF-β, PDGF-BB, TNF-α or lipopolysaccharide.

15. The method of claim 11 wherein the test compound is a putative anti-fibrotic agent, the method further comprising comparing the effect of the test compound with the effect of a known anti-fibrotic agent.

16. The method of claim 11 wherein the test compound is a putative pro-fibrotic agent, the method further comprising comparing the effect of the test compound with the effect of a known pro-fibrotic agent.

17. The method of claim 15 wherein the known pro-fibrotic agent is TGF-β, PDGF-BB, TNF-α or lipopolysaccharide.

18. The method according to claim 1 that is a method of treating a pancreatic fibrosis related disorder in a subject.

19. (canceled)

20. The method according to claim 18 wherein the PSC is in vitro and the PSC is administered to the subject.

21. The method according to claim 17 wherein the PSC is in vivo.

22. The method according to claim 6 that is a method of diagnosing the presence of pancreatic fibrosis in a subject or determining the prognosis of a subject having or being likely to develop pancreatic fibrosis and wherein the coding sequence encodes a marker molecule, the method further comprising:
a) detecting the expression level of the marker molecule, wherein the PSC is an in vitro PSC removed from the subject; and
b) comparing the expression level in the PSC from the subject with expression of a coding sequence encoding a marker molecule that is operably linked to a GFAP promoter in a pancreatic stellate cell that is not associated with pancreatic fibrosis.

23. A pancreatic stellate cell (PSC) comprising a nucleic acid molecule comprising a coding sequence operably linked to glial fibrillary acidic protein (GFAP) promoter, the GFAP promoter consisting essentially of the 2.2 kilobase region 5′ to a GFAP gene or a fragment thereof and the coding sequence encoding a marker molecule or a therapeutic molecule.

24. (canceled)

25. (canceled)

26. (canceled)

27. (canceled)


SEQUENCE LISTING (4 sequences)

SEQ ID NO: 1 - DNA, 1675 bp, Homo Sapien
gtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcat

SEQ ID NO: 2 - DNA, 1604 bp, Rattus norvegicus
cctgcagggcccactagtctgtaagctggaagtctggcagtgctgagctggccaaccccctcaggacctcctccttgtgcccactgaatgactcaccttggcatagacataatggtcaggggcgggcacacagcctgattcccgctgcactccaggcccccttcaatgctttccgagaagtccattgagctgggagcttgtactgcaccaagggctgacatcctggcagccagggatgaaagcagcccatggggctacccttgccgtatgcctcactggcggcagagaacaaggctctattcagcaaatgccctggagtagacaccagaagtccaagcatgggcagaggaaggcaggcgttgggggctggaggggagcagagctgtctgttttccagaagcccaagggtacagatggcgcctgggggggaactgagtggaggggatagatgggcctgagatctcaaacatcaacagcctcctccccaccaacgatgaaggtggaggttggtttcccagacctacatatcccccagagacctggtgtatgaaaattcaaaggaggtaagtctcctgagagaacggggggctcacaaatgaagccagctgtcttaccctatcaggacctacgtgcattccttctgtcctgccccctaaacacacagccagaggctcaaattgattctggagtcacaaagggggcttgaaaccccagccccccactcctgaactccaggaatgagaagatagtattggaggggttcagaggagagggctctgcacatctgttgagaatgggggtcccaggagagtgtaatttaggctgatcccggaggaagggaataggctcttcaagatcctagcatctcacaggcccacagagaagttcagagttggggcagccctggcttacaggctctaagaactggaggcagtttacccaacccagctgtgtgcatgctgtccctctctctgtctctgtctgtctctctctgtctctgtctctctgtgtgtgtgtgtgtgtgctcacacacgtgtgtgtttatcacacaaatgttcatgtgtgtgtacatacatgtgttgaggccagaggtcaacctcagacactgttgacttggttgtatgagataacatttccccctgggacctgggatttgccaattagtgtgacccaggaagcctacttattttcattcctcagcactgcagttacaagtatgcactgtcaaaccaggcctttttttttttttttttccaaaccaggccttttgtattcgctctgtggctagaacttgggtctccatgcttgacaggcaagcgatttatggactaagctgtttcctcggccctctcttgacccatttaccagaaatggggtttccttgatcaatggttaagccaggctggtgttcccaggaaacccttgactctgggtacagtgaccttggtggggtgagaagagttctctccatagctgggctggggcccagctccaccccctcaggctattcaatggggtgctgccaggaagtcaggggcagatccagtccagcccgtccctcaataaaggccctgacatcccaggagccagcagaagcagggcat

SEQ ID NO: 3 - DNA, 20 bp, Artificial Sequence
  Synthetic oligonucleotide primer
actccttcataaagccctcg

SEQ ID NO: 4 - DNA, 26 bp, Artificial Sequence
  Synthetic oligonucleotide primer
aactcgccgcacatctgaacttcagc